(19) United States
(12) Patent Application Publication
(10) Pub. No.: US 2011/0020841 A1
(43) Pub. Date: Jan. 27, 2011

(54) CENTROSOME AMPLIFICATION AS A BIOSENSOR FOR DNA DAMAGE

(75) Inventors: Ciaran Morrison, Galway (IE); Helen Dodson, Galway (IE); Emer Bourke, Galway (IE)

Correspondence Address: FOLEY & LARDNER LLP, 975 PAGE MILL ROAD, PALO ALTO, CA, 94304, US

(73) Assignee: National University of Ireland, Galway

(21) Appl. No.: 12/443,810

(22) PCT Filed: Oct. 5, 2007
(86) PCT No.: PCT/IE2007/000093

§ 371 Date: Jul. 30, 2010

(30) Foreign Application Priority Data
Oct. 5, 2006 (IE) 2006/0731

(51) Int. Cl. G01N 33/53

(52) U.S. Cl. 435/7.21

(57) ABSTRACT

The present invention provides an assay kit for identifying and/or monitoring genotoxic modulating agents comprising eukaryotic cells, characterised in that the cells stably express at least one labelled centrosome marker.

### BACKGROUND TO THE INVENTION

Toxicological screening is an important aspect of compound development. Regardless of whether the compound is destined for use as a drug, food additive, detergent, or other compound that may come into contact with humans or animals, it can be necessary to ascertain the genotoxic potential of the compound.

Traditional methods involved treating large numbers of animals with the compound in question, but these studies can be time consuming and expensive, as well as having the potential to raise ethical concerns in some observers. Various in vitro tests have therefore been proposed to limit the need for animal trials. Some of these tests, such as the Ames test and that disclosed in WO 98/38336, rely on the reaction of bacterial genomes to genotoxic stress and so the results are not always directly relevant for humans. U.S. Pat. No. 5,352,581 discloses an assay that uses yeast cells, which may also respond to genotoxic damage differently to human cells.

Numerous tests have been proposed to overcome the perceived shortcomings of the above-assays. WO 2004/034013, U.S. Pat. No. 4,794,074 and U.S. Pat. No. 5,096,808 disclose immunoassays and kits monitoring human exposure to genotoxic agents.

U.S. Pat. No. 5,932,418 relies on cytogenetic assays of teleost embryos taken at different developmental stages. JP 2,242,156 discloses an assay requiring DNA or RNA molecules to be fixed to a support medium. U.S. Pat. No. 5,229,265 discloses an assay based on micronucleated cells and flow cytometry. WO 2006/050124 discloses an assay that relies on the gene expression profile of cells following genotoxic stress.

EP 1,217,077 discusses microorganisms and mammalian cells that have been genetically modified to produce light when the presence of a test agent results in a mutation in the DNA of the microorganism or cell.

However, all of these assays present different advantages and disadvantages over each other. For example, some of these assays do not lend themselves well to automation or high throughput, whereas others require antibody reactions or other reagents or instruments to function. Furthermore, many of these assays rely on the reactions of whole cell populations, or are conducted upon dead cells, or cells that have undergone extensive contact with various additional reagents involved in the assay. There exists a need to provide a simple, relatively low-cost assay that can be used to easily identify genotoxic agents, and that can be easily scaled up for high throughput. There also exists a need to provide an assay that can examine the effect of genotoxic agents on living cells, and can be used to examine the effect on large numbers of cells, for high throughput, and can equally be used on individual cells.

WO 86/03007 discloses an assay procedure that combines the test sample with cultured cells and detects genotoxic substances by identifying structural changes in the cytoskeletal constituents in comparison to normal control cells. However, cytoskeletal alterations in a cell can arise for a number of reasons not related to genotoxicity, such as reactivity to cytokines or nutrients, cell motility or intra-cell communication, to name a few.

The inventors have previously shown by combining electron and light microscopy that centrosome amplification occurs in human cells after ionising radiation treatment. Similar results in Rad51-deficient chicken DT40 cells¹, irradiated mammalian cells^{2,3} and DNA topoisomerase II inhibitor-treated DT40 cells suggest that centrosome amplification is a general response to DNA damage.

Centrosomes are the principal microtubule organizing centers (MTOCs) in animal cells. They consist of two cylindrical centrioles embedded in an amorphous matrix of pericentriolar material^{4,5}. Centrosomes normally replicate only once per cell cycle in a tightly-regulated process, as two centrosomes are necessary to establish the bipolar mitotic spindle critical for cell division. Multiple centrosomes can lead to multipolar mitoses, potentially contributing to aneuploidy and genomic instability. Abnormal centrosome numbers have been observed in—p53- or p21-deficient cells^{6,7}, in cells with DNA repair deficiencies^{1,8}, in cells with telomere defects⁹ and in human papillomavirus-infected cells¹⁰. In an important early study, amplification of MTOCs was observed following irradiation of mouse cells, although EM showed that a high percentage of the resulting MTOC structures did not contain the paired centrioles and pericentrosomal material that comprise the normal centrosome³. Later work on irradiated human U2OS osteosarcoma cells demonstrated MTOC amplification after irradiation², although the structure of these MTOCs was not determined.

It is also known that inhibition of the DNA damage-responsive Chk1 kinase using the drug UCN-01 or RNAi suppresses DNA damage-centrosome amplification and that abrogation of Chk1 function by disruption of the Chk1 gene completely abolishes such a response¹¹, demonstrating that Chk1 is the principal effector through which DNA damage decouples the chromosome and the centrosome cycles in vertebrate cells.

### SUMMARY OF THE INVENTION

The present invention provides an assay kit for identifying and/or monitoring genotoxic modulating agents, the assay kit comprising eukaryotic cells, characterised in that the cells stably express at least one labelled centrosome marker.

A genotoxic modulating agent may be defined as an agent that effects the genotoxic damage on a cell. The genotoxic modulating agent may directly or indirectly cause, increases, potentiates, exacerbates, reduces, limits, treats or cures genotoxicity or genotoxic damage on a cell. In some embodiments, one or more of the inventions may be restricted to genotoxic agents that are deleterious to the cell; i.e., that increase genotoxicity, (for example, either directly or indirectly), (for example by potentiating the effect of a second agent or stress). In some embodiments, the invention may be related to genotoxic modulating agents that limit, reduce, treat, salve or cure the effects of a genotoxic agent or stress, either directly or indirectly. Thus, the assays of the invention can be used to detect genotoxic agents as well as detecting possible treatments or ameliorating compounds to reduce the effects of that genotoxic agent or stress or other genotoxic agent or stress. Naturally, where aspects of the invention are described in respect of one aspect of a genotoxic modulating agent (such as deleterious agents that cause DNA damage), the invention also relates to the aspect of the invention that relate to ‘positive’ agents, that ameliorate or potentiate against DNA damage.

The centrosome marker may be selected so as to permit definitive individual quantification of the centrosome. The centrosome marker may also be selected to provide definitive visualisation of the centrosomes

The cells may be living. One of the advantages of some embodiments of the present invention is that the assay may be performed on living cells as well as, or as an alternative to, fixed cells. In some embodiments of the invention, the cells may be modified cells. Modified calls are cells whose natural response to genotoxic stress and agents has been altered. This may, for example, be as a result of genetic modification, either intentional or unintentional, gene silencing (for example, by siRNA), viral infection, bacterial infection, radiation, chemical or environmental mutagenesis. The assay can therefore be used to assess genotoxic damage or stress caused intentionally or unintentionally by viral or bacterial infection or other genotoxic stresses. The assay can also be used to assess the effect of compounds or treatments or strategies for modifying, treating, preventing, limiting or increasing the effect of genotoxic stress.

The present invention provides an assay kit that utilises centrosome amplification upon exposure to genotoxic agents by relying on a visualisation of the centrosome. In some embodiments of the invention, the centrosome marker is centrin 1. One of the advantages of centrin-1 is that it is a structural component of the centriole. However, in some embodiments, the invention provides for the use of other centrosome markers, such as one or more selected from the group consisting of gamma-tubulin, centrin3 and Aurora A.

The assays of the present invention may also comprise at least one labelled nuclear marker. The nuclear marker may be selected so as to permit definitive visualisation of the nucleus throughout the cell-cycle. The nuclear marker allows individual cells to be visualised in order to quantify the number of centrosomes per cell and/or to score them either quantitatively, or qualitatively. Moreover, the use of a nuclear marker permits additional data to be obtained by the assay, such as cell cycle stage, mitotic fate and formation of micronuclei. In some embodiments, different labels can be applied to the centrosome markers (which may be the same or different) of different cell populations within the same assay, for example, in order to assess how different cells react to the same conditions when the cells are in communication with each other. In some embodiments of the invention, the labelled nuclear marker is Histone 2B (H2B). H2B allows chromatin to be visualised during long term imaging experiments. Some vital DNA dyes can be toxic to a cell eventually; this disadvantage is overcome using tagged Histone 2B. Histone 2B also allows the visualisation of mitotic and/or apoptotic cells, which indicates the cellular outcomes of the test treatment—death, survival, aberrant division, etc. In some embodiments the invention provides for the use of other nuclear markers, such as one or more selected from centromere or telomere markers to view individual chromosomes, or DNA damage-response proteins that form nuclear foci after DNA damage, thus providing an additional datum in the analysis of the DNA repair capacity of a cell. The centromere marker may be one or more selected from the group consisting of Cenp-H and Survivin. The telomere marker may be Trf1. The DNA damage pathways marker may be one or more selected from the group consisting of: Nbs1 and PCNA. In some embodiments, particularly those not using live cells, DAPI or Hoechst 33342 may be used. These markers may be used alone or in combination with each other and/or Histone 2B.

The assay kits of the present invention may also comprise at least one labelled cell-cycle marker. The cell-cycle marker may be selected so as to permit definitive identification of at least one distinct stage during the cell-cycle. The cell-cycle marker allows identification of which stage of the cell cycle the genotoxic agents exert their various effects, amongst other uses e.g., identification of any cell cycle delays imposed by treatment, aberrant timing of cell cycle events after treatment. Different cell cycle markers may allow the definition of a genotoxin-induced cell cycle arrest in G1/S (cyclin A localisation, PCNA focal pattern), G2 (nuclear localisation of cyclin B) or M (H2B to determine chromosome condensation) phase, which may be of benefit in certain applications. Some embodiments of the invention provide for the use of other cell-cycle markers, such as one or more selected from the group consisting of PCNA, Cyclin A, Cyclin E, Cyclin B.

Histone 2B (H2B) is always present in the nucleus and is always chromosome-associated, but mitotic chromosome condensation allows H2B to identify mitosis. Thus, in some embodiments, H2B may be used as a cell-cycle marker or a marker for mitosis.

The centrosome markers may be labelled by fluorescent tagging. The nuclear markers may be labelled by fluorescent tagging. The cell-cycle markers may be labelled by fluorescent tagging. Moreover, one or more of the centrosome, nuclear and cell-cycle markers may be labelled by any labelling approach which permits rapid visualization of a large number of cells, different populations of cells, cells exposed to different agents or stresses, and applications in high throughput screening assays. Such labelling methods may be selected from one or more of the group consisting of luminescent tagging, radiolabelling, and various epitope tags.

Some embodiments of the invention relate to an assay kit for identifying and/or monitoring the impact of genotoxic agents or stresses (or teratogenic agents or stresses) comprising living eukaryotic cells, characterised in that the cells stably express at least one fluorescently-tagged centrosome marker and at least one fluorescently-tagged nuclear marker.

The cell line may be a tissue-culture cell line. The cells may be stem cells. The cells may be adult stem cells. The cells may be embryonic stem cells. In some embodiments, the cells may be selected for their appropriateness to detect the response of a particular cell or species type to a particular agent or stress. The cells may be human in origin. The cells may be chicken in origin. The cells may be murine in origin. In some assays, the cells may be of mammalian origin. The cells may be of origin selected from the group consisting of human, chimpanzee, monkey, mouse, rat, hamster, guinea pig, rabbit, chicken, cattle, sheep, goat, horse, donkey, dog and cat. The cell lines may be selected from the group consisting of human lymphoblastoid cells, Jurkat T-cell leukaemia, Hct116 colon carcinoma and U2OS osteosarcoma. The cells may be grown in 96-well, 384-well (or other multiple-well plates) for ease of automation and also to keep reagent volumes and costs low.

The present invention also provides methods for the generation of cell lines suitable for an assay to detect genotoxic agents or stresses, comprising transfecting one or more eukaryotic cell lines such that the cells can express at least one labelled centrosome marker and/or at least one labelled nuclear marker. The cells may also express at least one labelled nuclear marker as provided in the assay herein described.

In use, a count of over about 2 centrosomes per cell may indicate the effect of a genotoxic agent or stress. Under some conditions, a low level of background may be observable, but the skilled person will have little difficulty in allowing for this and compensating accordingly. The effect may be detected by means of fluorescence microscopy. The fluorescent microscopy read-out value can be calibrated to indicate how many centrosomes per cell are present.

The invention also comprises a method of identifying and/or monitoring the impact of genotoxic agents or stresses (or teratogenic agents or stresses) comprising the assay as herein described. The assay can be expanded to monitor the effect of successive exposure to the same or different agents over time, as well as monitoring the effect or otherwise of potential treatments.

The invention also comprises a method for screening for inhibitors of centrosome amplification agents or agents that inhibit centrosome amplification, comprising treating eukaryotic cells with candidate stresses or agents, characterised in that the cells stably express at least one labelled centrosome marker. The cells may also express at least one labelled cell cycle marker. The cells may also express at least one labelled nuclear marker. The various options and possibilities for the method and markers are as described herein. The centrosome amplification agent may be Chk1 kinase.

In some embodiments, the present invention provides a method for using live cell imaging of centrosomes as a tool to monitor DNA damage and/or Chk1 signalling. In some embodiments, the present invention provides human cell lines (Hct116 colon carcinoma and U2OS osteosarcoma) that stably express fluorescently-tagged versions of the centrosome marker, centrin-1 in various combinations with fluorescent versions of the cell cycle markers PCNA, Cyclin A, Cyclin E and Cyclin B and the nuclear marker, Histone 2B. Some embodiments of the invention utilise adherent cells. Non-adherent cells move in suspension and are therefore more difficult to screen individually. Some adherent cell types can grow over each other, thereby making individual assessment difficult where this is permitted to occur. The U2OS cells are adherent and remain separate in culture and thus can be imaged very easily and then scored. In some embodiments of the invention, differences in the absolute numbers of centrosomes induced by given genotoxic agents may be observable between different cell lines or cell types.

The fluorescent centrin1/histone 2B—expressing lines represent cells with fluorescent centrosomes and chromosomes. The present invention can utilise live-cell microscopy analysis of centrosome amplification after DNA damage as a biomarker for genotoxic stress, for DNA-damaging drugs.

Some embodiments of the invention provide a method for identifying candidate genes involved in centrosome amplification and/or genotoxic stress response. The method may further comprise performing an assay substantially as herein described and further coupling the assay with gene repression of one or more candidate genes to determine whether the one or more candidate genes, alone or in combination, are involved in one or both of involved in centrosome amplification and genotoxic stress response. The gene repression may comprise siRNA gene repression.

It will be understood that aspects of the assay kits as described herein may be suitable for use with the various assays and methods described herein, and certain aspects and features described with reference to of the various assays and methods may be suitable for inclusion with kits of the invention.

### Materials and Methods

### Cloning

Centrin-1-encoding sequence was cloned into pEGFP-N1 and pEGFP-C1 (Invitrogen™) pmRFP-N1 and pmRFP-C1 were cloned by replacement of the GFP coding sequence in pEGFP-N1 and pEGFP-C1, respectively, with sequence encoding monomeric Red Fluorescent Protein (mRFP). cDNA sequence encoding histone 2B was generated by RT-PCR from RNA from Jurkat cells and cloned into pmRFP-N1. Cloning protocols used standard methods. All constructs were verified by DNA sequencing.

### Cell Culture, Transfection and Genotoxic Treatment

Human U2OS cells were obtained from the ATCC and cultured according to ATCC specifications at 37° C. in DMEM. Human lymphoblastoid cells GM07521 (apparently normal) were obtained from Coriell Cell Repositories and were cultured according to Coriell's specifications in RPMI 1640. Transfections were performed using lipofectamine 2000™ (Invitrogen™) and transfected clones were selected under neomycin or puromycin. Individual clones were picked using cloning rings, expanded and analysed by microscopy for expression of the transgene(s) of interest. Gamma-irradiation was performed using a¹³⁷Cs source (Mainance Engineering). Caffeine (Sigma™) was dissolved in water at 200 mM and treatment involved a 1 hour preincubation of cells with 2 mM caffeine prior to further experimentation.

### Microscopy

Cells were grown on sterile coverslips and were then viewed live after a single phosphate-buffered saline (PBS) wash, or fixed with either 4% paraformaldehyde in PBS for 10 minutes at room temperature. Where immunofluorescence was necessary, cells were then washed 3 times in PBS before blocking in PBS/1% bovine serum albumin. Primary antibodies used against γ-tubulin were mouse GTU88 at 1:100 and rabbit T-3559 at 1:1000 (both from Sigma). Primary antibody against phospho-histone H2AX was mouse monoclonal JBW103 (Upstate) and was used at 1:1000. Primaries were diluted in blocking solution with incubations being performed for 1 hour at 37° C. The cells were then washed 3 times in PBS before incubation with appropriate secondary antibodies (fluorescein isothiocyanate (FITC) and Texas Red-coupled anti-mouse and anti-rabbit antibodies from Jackson Laboratories™ diluted 1:200 in blocking buffer and Alexa 488-coupled anti-rat antibodies from Molecular Probes™ diluted 1:1000 in blocking buffer). Where a DNA counterstain was required, samples were washed and counterstained with DAPI prior to mounting in Vectashield™(Vector Laboratories“). Cell counting and imaging was performed using an Olympus™ BX51 microscope, 100× objective, N.A. 1.35 using Openlab software (Improvision”).

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

### Results and Discussion

Preliminary data using ionising radiation to induce DNA damage demonstrated the feasibility of aspects of the present invention. Following extensive analysis of the role of Chk1 in DNA damage-induced centrosome amplification using fixed cells, it is believed that suppression of radiation-induced centrosome amplification by Chk1 inhibitors (genetic or pharmacological) offer a novel biomarker for Chk1-targeting drugs, which can also be analysed by microscopy. The ability to automate this assay (using 96-well, 384-well or other multi-well format tissue culture and automated microscopy) provides for the present invention to be used in screening procedures.

In some embodiments, the present invention seeks to employ centrosome amplification, which occurs after DNA damage, as a means to monitor the impact of genotoxic agents or stresses on living tissue culture cells. As shown in FIG. 1A clones of the human U2OS line were generated that stably express fluorescently-tagged centrin, a centrosome marker, and fluorescently-tagged histone 2B, a marker for the chromosomes.

To confirm the identity of the structures observed with the tagged centrin construct used here, immunofluorescence microscopy was performed with antibodies to the centrosome component, gamma-tubulin. As shown in FIG. 1B gamma-tubulin co-localised with the fluorescently-tagged centrin, confirming the centrosomal identity of the centrin-containing foci. Fixed (FIG. 2A) and live-cell (FIG. 2B), microscopy to detect centrin was used to quantitate the number of centrosomes that are contained in a given cell. As shown in an analysis performed in living U2OS cells, the number of cells that contain >2 centrosomes increases with time after ionising radiation treatment, along with a detectable increase also occurring in the number of cells with aberrant centrosome structures (FIG. 2C). To confirm that the ionising radiation protocol used results in cellular DNA damage, the formation of nuclear foci of phosphorylated histone H2AX (gamma-H2AX) was used as a marker for cells with DNA damage (FIG. 3).

The present invention can thus provide a system to visualise centrosome amplification in living human tissue culture cells. In published work¹, it has been shown that centrosome amplification serves as a marker for DNA damage, induced by irradiation, DNA repair deficiencies or DNA topoisomerase II inhibition. The present invention provides a system that may be used in testing DNA damaging agents. The present invention also provides an assay that can test agents that inhibit DNA damaging agents or which reverse the effects of DNA damaging agents.

In some aspects, the present invention demonstrates that Chk1 kinase is required for centrosome amplification to occur as a consequence of DNA damage induced by irradiation or by DNA topoisomerase II inhibition. As centrosome amplification is a highly penetrant phenotype resulting from DNA damage, the present invention provides a live cell screen for inhibitors of Chk1 kinase activity and/or other enzymes/enzyme systems involved in inducing, inhibiting or reversing DNA damage. By way of example, if a treatment impedes Chk1 activity, the inability to undergo centrosome amplification can be detected, as shown in FIG. 4, where caffeine treatment, which blocks Chk1 activation by inhibiting its upstream activators, suppresses centrosome amplification.

### BRIEF DESCRIPTION OF THE DRAWINGS

FIG. 1: Generation of fluorescently-tagged centrosome markers.

A. Visualisation of centrosomes and chromosomes in (upper panels) interphase and (lower panels) mitotic U2OS cells expressing GFP-centrin1 (green) and H2B-RFP (red). Cells were imaged after fixation in 4% parafromaldehyde, scale bar is 10 μm.

B. Centrosomal localisation of GFP-centrin1. U2OS cells expressing GFP-centrin1 (green) were stained with anti-γ-tubulin (blue) following fixation in methanol. Scale bar is 10 μm.

FIG. 2: DNA damage-induced centrosome amplification

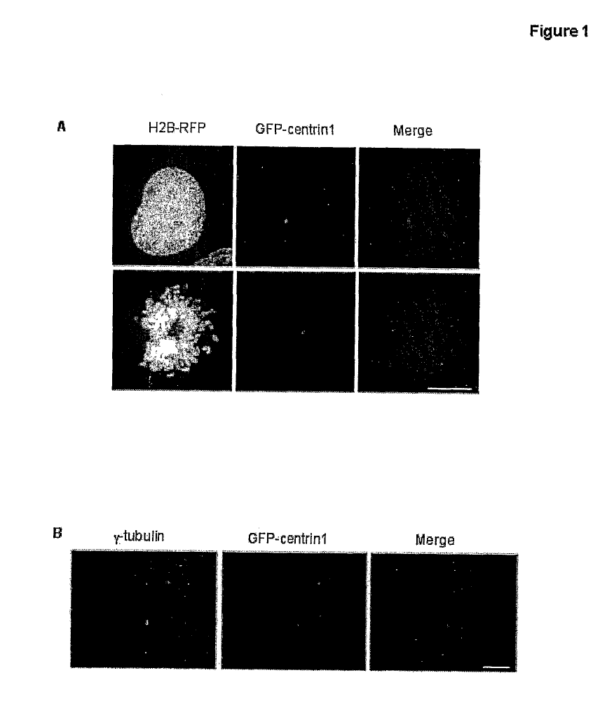

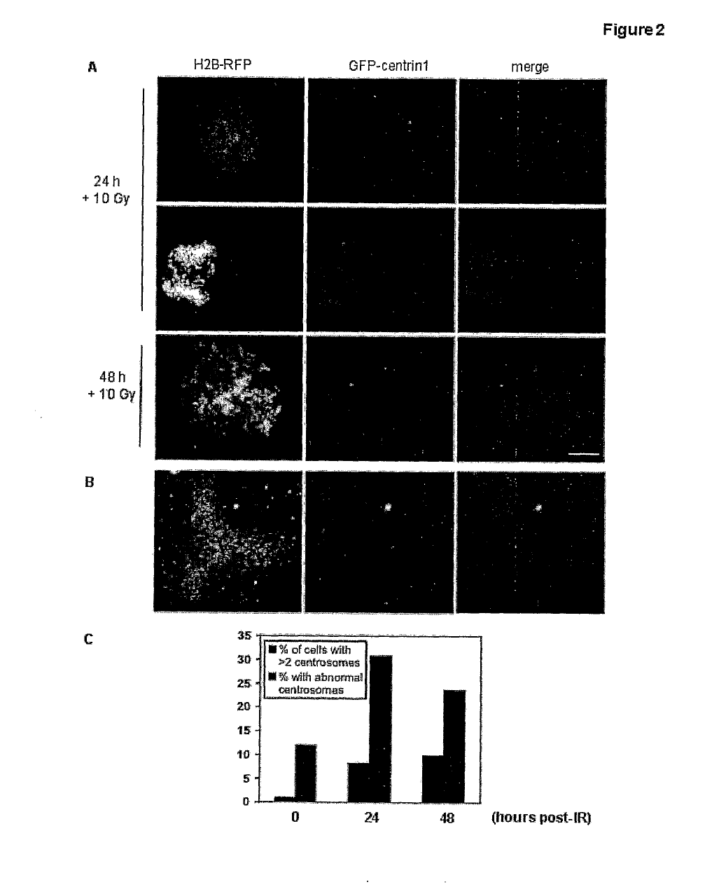

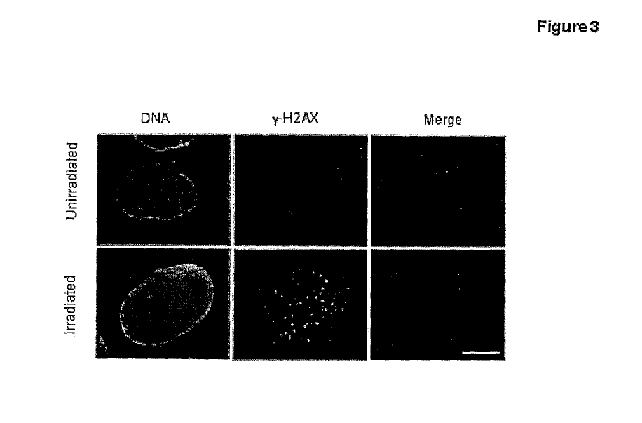

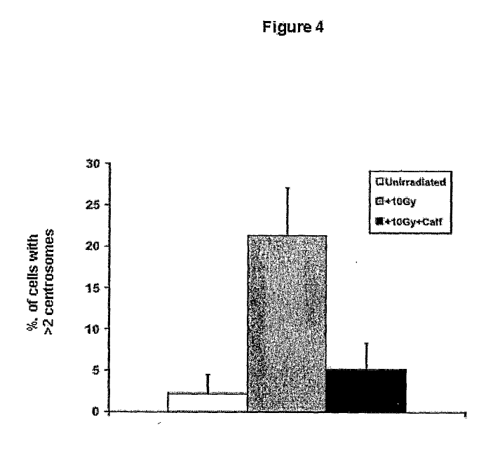

A. Examples of centrosome aberrations after 10 Gy ionising radiation treatment visualised in fixed U2OS cells expressing GFP-centrin-1 and H2B-RFP. Scale bars are 10 μm.

B. Centrosome abnormalities observed in a live U2OS cell expressing GFP-centrin-1 (green) and H2B-RFP (red) after 10 Gy ionising radiation treatment.

C. Histogram showing quantitation of centrosome abnormalities in live U2OS cells at indicated times following 10Gy ionising radiation. Cells were washed in PBS prior to fluorescence microscopy and were counted as abnormal when having >2 centrosomes or when having up to 4 aberrantly-separated centrioles. At least 100 cells per timepoint were counted.

FIG. 3: Confirmation of DNA damage induction

Immunofluorescence micrograph showing ionising radiation induction of nuclear foci of phosphorylated histone H2AX (γ-H2AX; red), which form at sites of DNA damage. 10 Gy gamma-irradiation was performed 90 minutes prior to fixation. DNA is shown in blue and scale bar is 10 μm.

FIG. 4: Caffeine repression of centrosome amplification

Quantitation of human lymphoblastoid cells with multiple centrosomes before and 48 h after IR in the presence or absence of caffeine, as indicated. Centrosomes were counted by immunofluorescence microscopy of γ-tubulin spots. Data were obtained from at least 500 cells per experiment and histograms show the mean +s.d. of results from 3 separate experiments, performed blind.

### REFERENCES

1. Dodson, H., Bourke, E., Jeffers, L. J., Vagnarelli, P., Sonoda, E., Takeda, S., Earnshaw, W. C., Merdes, A. & Morrison, C. Centrosome amplification induced by DNA damage occurs during a prolonged G2 phase and involves ATM. Embo J 23, 3864-73 (2004).
2. Sato, N., Mizumoto, K., Nakamura, M. & Tanaka, M. Radiation-induced centrosome overduplication and multiple mitotic spindles in human tumor cells. Exp Cell Res 255, 321-6 (2000).
3. Sato, C., Kuriyama, R. & Nishizawa, K. Microtubule-organizing centers abnormal in number, structure, and nucleating activity in x-irradiated mammalian cells. J Cell Biol 96, 776-82 (1983).
4. Doxsey, S., McCollum, D. & Theurkauf, W. Centrosomes in Cellular Regulation. Annu Rev Cell Dev Biol (2005).
5. Doxsey, S. Re-evaluating centrosome function. Nat Rev Mol Cell Biol 2, 688-98 (2001).
6. Mantel, C., Braun, S. E., Reid, S., Henegariu, O., Liu, L., Hangoc, G. D. Broxmeyer, H. E. p21(cip-1/waf-1) deficiency causes deformed nuclear architecture, centriole overduplication, polyploidy, and relaxed microtubule damage checkpoints in human hematopoietic cells. Blood 93, 1390-8 (1999).
7. Fukasawa, K., Choi, T., Kuriyama, R., Rulong, S. & Vande Woude, G. F. Abnormal centrosome amplification in the absence of p53. Science 271, 1744-7 (1996).
8. Griffin, C. S., Simpson, P. J., Wilson, C. R. & Thacker, J. Mammalian recombination-repair genes XRCC2 and XRCC3 promote correct chromosome segregation. Nat Cell Biol 2, 757-61 (2000).
9. Guiducci, C., Cerone, M. A. & Bacchetti, S. Expression of mutant telomerase in immortal telomerase-negative human cells results in cell cycle deregulation, nuclear and chromosomal abnormalities and rapid loss of viability. Oncogene 20, 714-25 (2001).
10. Duensing, S., Lee, L. Y., Duensing, A., Basile, J., Piboonniyom, S., Gonzalez, S., Crum, C. P. & Munger, K. The human papillomavirus type 16 E6 and E7 oncoproteins cooperate to induce mitotic defects and genomic instability by uncoupling centrosome duplication from the cell division cycle. Proc Nati Acad Sci USA 97, 10002-7 (2000).
11. Bourke, E., Merdes, A., Cuffe, L., Dodson, H., Zachos, G., Walker, M., Gillespie, D. & Morrison, C. Chk1 controls DNA damage-induced centrosome amplification. J. Cell Biol. submitted (2006).

1-11. (canceled)

12. A method of detecting Chk1 signalling and/or agents that inhibit, reduce or reverse Chk1 activity comprising a test pool of one or more cells and a control pool of one or more cells, wherein the control pool and the test pool comprise eukaryotic cells stably expressing at least one labelled centroseome marker the method comprising treating the test pool with at least one candidate agent, and comparing numbers of centrosomes per cell in the test pool and the control pool, wherein a difference in the numbers of centrosomes per cell between the test pool and the control pool is indicative of Chk1 signalling and/or agents that inhibit, reduce or reverse Chk1 activity.

13. A method of detecting Chk1 signalling and/or agents that inhibit, reduce or reverse Chk1 activity comprising a test pool of one or more cells and a control pool of one or more cells, wherein the one or more cells of the control pool and the test pool stably express at least one labelled centrosome marker, the method comprising treating the test pool with at least one candidate agent, and comparing numbers of centrosomes per cell in the test pool and the control pool, wherein a difference in the numbers of centrosomes per cell between the test pool and the control pool is indicative of Chk1 signalling and/or agents that inhibit, reduce or reverse Chk1 activity.

14. A method as claimed in claim 12 or 13 wherein the centrosome number is detected by means of fluorescence microscopy.

15. A method as claimed in claim 12 or 13, wherein a count of over 2 centrosomes in a particular cell indicates Chk1 signalling and/or agents that inhibit, reduce or reverse Chk1 activity on the particular cell.

16. A method as claimed in claim 12 or 13, wherein the cells are living.

17. A method as claimed in claim 12 or 13, wherein the cells further express at least one nuclear marker.

18-22. (canceled)

23. A method as claimed in claim 14, wherein a count of over 2 cenctrosomes in a particular cell indicates Chk1 signalling and/or agents that inhibit, reduce or reverse Chk1 activity on the particular cell.

24. A method as claimed in claim 14, wherein the cells are living.

25. A method as claimed in claim 15, wherein the cells are living.

26. A method as claimed in claim 14, wherein the cells further express at least one nuclear marker.

27. A method as claimed in claim 15, wherein the cells further express at least one nuclear marker.

28. A method as claimed in claim 16, wherein the cells further express at least one nuclear marker.
